(19) United States
(12) Patent Application Publication
(10) Pub. No.: US 2022/0127069 A1
(43) Pub. Date: Apr. 28, 2022

(54) BEVERAGE CAPSULE

(71) Applicant: Lify Company Limited, Hong Kong (HK)

(72) Inventors: Kong Wai Lee, Hong Kong (HK); Tak Chi Lee, Hong Kong (HK)

(21) Appl. No.: 17/427,677

(22) PCT Filed: Jan. 24, 2020
(86) PCT No.: PCT/CN2020/074046

§ 371 Date: Aug. 2, 2021

(30) Foreign Application Priority Data
Feb. 1, 2019 (HK) 19101852.4
Jan. 3, 2020 (HK) 32020000971.2

(51) Int. Cl. B65D 85/804; A23F 3/34; A23F 3/14; A61K 9/00

(52) U.S. Cl. CPC B65D 85/8055; B65D 85/8061; A61K 9/0095; A23F 3/14; A23F 3/34

(57) ABSTRACT

The present patent application relates to a beverage capsule, which includes a bracket, the bracket has a first opening and a second opening, the bracket includes an inner sidewall and a plurality of partition plates located on an inner side of the inner sidewall, the partition plates divide the columnar space evenly into a plurality of storage spaces, the storage spaces are filled with a brewing material, and further includes a first filter paper and a first sealing film for covering the first opening, and a second filter paper and a second sealing film for covering the second opening, a second end of the bracket has a plurality of end plates, the bracket further includes an outer sidewall, the outer sidewall first extends outward from one end of the inner sidewall and then extends to another end of the inner sidewall, an annular groove is formed between the inner sidewall and the outer sidewall, the bracket further includes a plurality of rib plates formed by extending outward from an outer surface of the inner sidewall. In the beverage capsule of the present patent application, since the bracket has partition plates and end plates, the structure's load-bearing capacity is more reasonable and is not easily deformed, it provides a space for expansion of the brewing material and will not affect the brewing effect caused by inability to expand.

### TECHNICAL FIELD

The present patent application relates to beverage capsules, and more specifically to a beverage capsule suitable for the brewing of materials such as Chinese medicine in pieces, herbal materials or tea leaves.

### BACKGROUND TECHNOLOGY

Since herbal materials, tea leaves and other materials cannot be ground into powder for direct infusion, they usually take a long time to infuse, and there is a problem that the nutrients of these raw materials cannot be extracted efficiently. Chinese medicinal preparations usually require a variety of Chinese medicine pieces to be matched, a user needs to mix various Chinese medicine pieces and then soaks or boils them for a long time, and it cannot guarantee an effective extraction of the effective ingredients, and causes great waste.

For some powdery soluble beverages, such as coffee, soy milk, fruit-flavored granules, etc., a form of capsules can be used to encapsulate the beverage raw material in a capsule for convenient storage and use. However, since Chinese medicine pieces, herbal materials and tea leaves are usually in granular form, pieces, and slices, they cannot be made into powder like coffee, and the powder form will also affect the extraction effect of Chinese medicine pieces, herbal materials and tea leaves. If Chinese medicine pieces, herbal materials, tea leaves and other materials are encapsulated in pieces in a traditional capsule, it causes accumulation and blockage easily, affects liquid flow in the capsule, and cannot sufficiently soak and extract the effective ingredients in these Chinese medicine pieces, herbal materials and tea, water cannot even flow in or out, which seriously affects the brewing effect. Therefore, there is an urgent need to develop a beverage capsule suitable for Chinese medicine in pieces, herbal materials and tea leaves.

Patent Application Contents

An objective of the present application is to provide a beverage capsule, which is especially suitable for the brewing of materials such as Chinese medicine in pieces, herbal materials and tea leaves, as well as for the brewing of coffee and other beverages.

The beverage capsule provided by the present application includes a bracket, the bracket has a first opening and a second opening, the bracket includes an inner sidewall and a plurality of partition plates located on an inner side of the inner sidewall, the inner side of the inner sidewall forms a columnar space, the first opening is formed at one end of the columnar space, the second opening is formed at another end of the columnar space, the partition plates divide the columnar space evenly into a plurality of storage spaces, the storage spaces are filled with a brewing material, a first end of the bracket is provided with a first filter paper and a first sealing film for covering the first opening, the first sealing film is located on an outer side of the first filter paper, a second end of the bracket is provided with a second filter paper and a second sealing film for covering the second opening, the second sealing film is located on an outer side the second filter paper;

the partition plates extend from a surface of the inner side of the inner sidewall to a central axis of the columnar space, or the partition plates extend from the surface of the inner side of the inner sidewall to the central axis of the columnar space and converge at the central axis;
the second end of the bracket has a plurality of end plates formed by extending inward from an end face of the inner sidewall, each of the end plates corresponds to one of the partition plates and is connected to the partition plate in one-piece, the end plate is perpendicular to the partition plate.

According to an embodiment of the beverage capsule of the application, the bracket further includes an outer sidewall, the outer sidewall first extends outward from one end of the inner sidewall and then extends to another end of the inner sidewall, an annular groove is formed between the inner sidewall and the outer sidewall, an annular block piece is further formed on an outer side of the outer sidewall.

According to an embodiment of the beverage capsule of the application, the bracket further includes a plurality of rib plates being formed by extending outward from an outer surface of the inner sidewall, each of the rib plates corresponds to one of the partition plates, the plurality of rib plates is located in the annular groove.

According to an embodiment of the beverage capsule of the application, the brewing material is a Chinese medicine in pieces, an herbal material or tea, and the herbal material includes one or more of a root, a stem, a leaf, a flower and a fruit of a plant.

According to an embodiment of the beverage capsule of the application, the first sealing film and the second sealing film are fiber films.

According to an embodiment of the beverage capsule of the application, the first sealing film and the second sealing film are translucent films.

According to an embodiment of the beverage capsule of the application, a portion of the outer sidewall located above the annular block piece and a portion of the outer sidewall located below the annular block piece have matching tapered surfaces.

The beverage capsule implemented in the present patent application has the following beneficial effects: since the bracket has partition plates and end plates, the structure's load-bearing capacity is more reasonable and is not easily deformed, it provides a space for expansion of the brewing material and will not affect the brewing effect caused by inability to expand.

### DESCRIPTION OF THE DRAWINGS

The present patent application will be further explained below in conjunction with the drawings and embodiments. In the drawings:

FIG. 1 is an exploded schematic view of an embodiment of a beverage capsule according to the present application;

FIG. 2 is an exploded schematic view from another angle of an embodiment of the beverage capsule according to the present application;

FIG. 3 is a perspective schematic view of an embodiment of the beverage capsule according to the present application;

FIG. 4 is a perspective schematic view from another angle of an embodiment of the beverage capsule according to the present application;

FIG. 5 is a schematic top view of an embodiment of the beverage capsule according to the present application;

FIG. 6 is a schematic bottom view of an embodiment of the beverage capsule according to the present application;

FIG. 7 is a schematic top view of the bracket in an embodiment of the beverage capsule according to the present application;

FIG. 8 is a schematic bottom view of the bracket in an embodiment of the beverage capsule according to the present application;

FIG. 9 is a schematic longitudinal cross-sectional view of the bracket in an embodiment of the beverage capsule according to the present application;

FIG. 10 is another schematic longitudinal cross-sectional view of the bracket in an embodiment of the beverage capsule according to the present application;

FIG. 11 is a schematic diagram of the beverage capsule shown in FIG. 3 being loaded into a brewing system in a forward direction;

FIG. 12 is a schematic diagram of the beverage capsule shown in FIG. 3 being loaded into the brewing system in a reverse direction;

FIG. 13 is an exploded schematic diagram of another embodiment of the beverage capsule according to the present patent application;

FIG. 14 is an exploded schematic view of the beverage capsule shown in FIG. 13 from another angle;

FIG. 15 is a perspective schematic view of the bracket of the beverage capsule shown in FIG. 13;

FIG. 16 is a perspective schematic view of the bracket shown in FIG. 15 from another angle;

FIG. 17 is a perspective schematic view of the beverage capsule shown in FIG. 13.

### SPECIFIC MODES OF IMPLEMENTATION

In order to have a clearer understanding of the technical features, purposes and effects of the present patent application, a specific implementation of the present patent application will now be described in detail with reference to the drawings.

The following describes in detail the embodiments of a beverage capsule of the present patent application. Examples of these embodiments are shown in the drawings, wherein the same or similar reference numerals indicate the same or similar elements or elements with the same or similar functions.

In the description of the beverage capsule in the present patent application, it should be understood that the terms “front”, “rear”, “upper”, “lower”, “upper end”, “lower end”, “upper portion”, “lower portion”, etc. that indicate the orientation or positional relationship are based on the orientation or positional relationship shown in the drawings, and are only used for the convenience of describing the present patent application and simplifying the description, and do not indicate or imply that the device or element referred to must have a specific orientation or a specific orientation The structure and operation cannot therefore be understood as a limitation of the present patent application. In addition, the terms “first”, “second”, etc. are only used for descriptive purposes, and cannot be understood as indicating or implying relative importance.

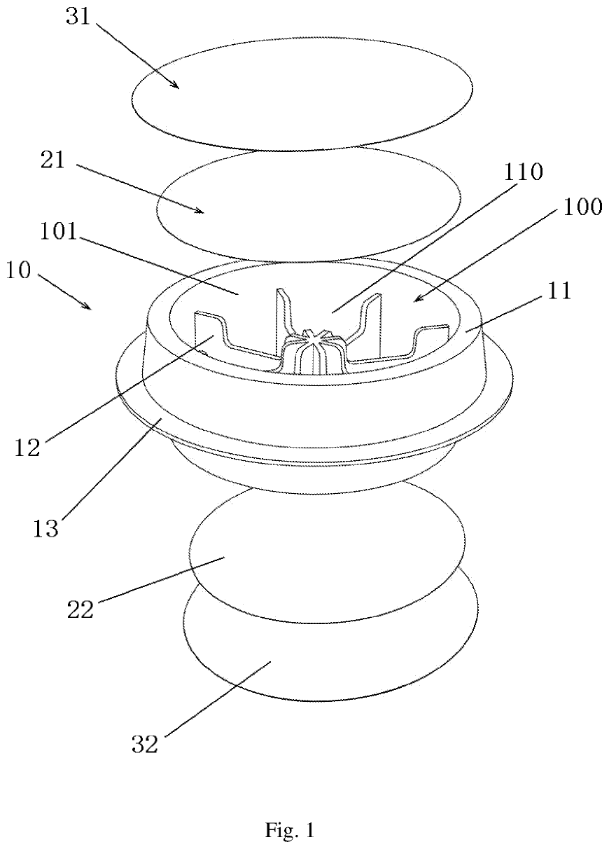

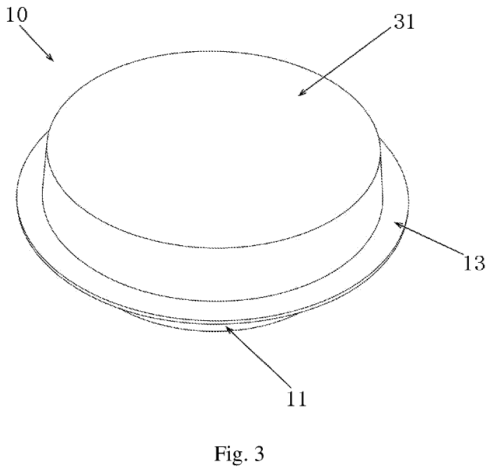

As shown in FIG. 1 to FIG. 12, it is an embodiment of a beverage capsule according to the present application. The beverage capsule in this embodiment includes a bracket 10, the bracket 10 may be made of a food-grade material, or a material such as plastic. The bracket 10 has a first opening 101 and a second opening 102, the bracket 10 includes an inner sidewall 11 and a plurality of partition plates 12 located on the inner side of the inner sidewall 11, the inner sidewall 11 and the partition plates 12 can be integrally formed, such as by integral injection molding, the partition plates 12 extend from the inner side surface of the inner sidewall 11 to a central axis of a columnar space 100, the inner side of the inner sidewall 11 forms the columnar space 100, the first opening 101 and the second opening 102 are respectively located at both ends of the columnar space 100, the partition plates 12 divide the columnar space 100 into a plurality of storage spaces 110, the storage spaces 110 are filled with a brewing material (not shown). The brewing material can be a Chinese medicine in pieces, an herbal material or tea leaves, the herbal material includes one or more of a root, a stem, a leaf, a flower and a fruit of a plant, tea leaves can be various kinds of tea leaves sold in the market, the Chinese medicine in pieces can be a variety of well-prepared Chinese medicines. The brewing material can also be coffee and other beverages.

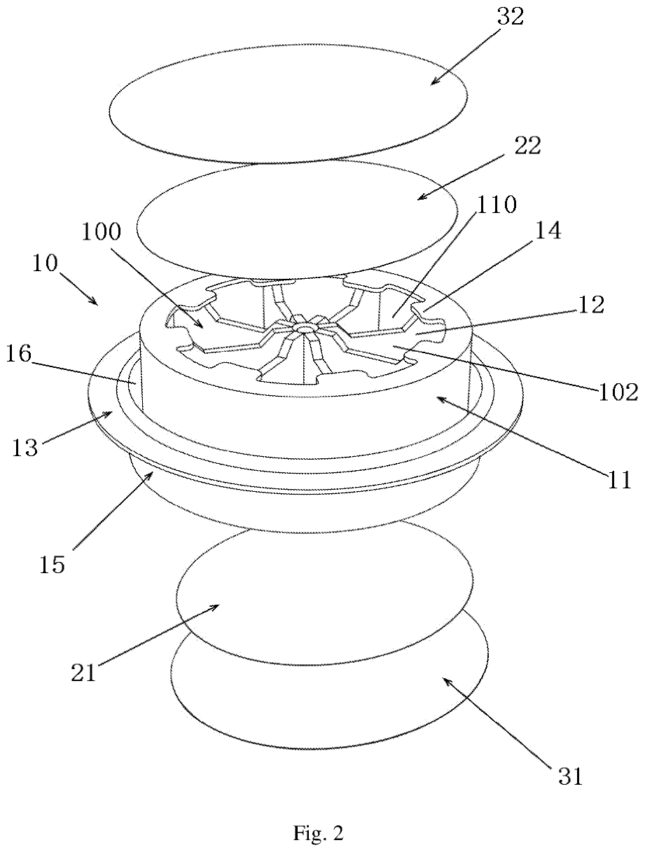

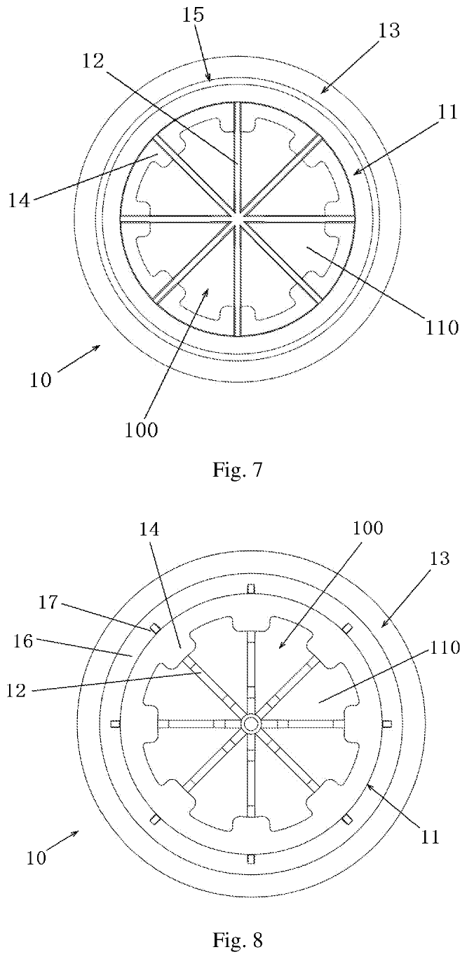

Referring to FIG. 1, FIG. 2, FIG. 7 and FIG. 8, in the present embodiment, eight partition plates 12 are formed on the inner side surface of the inner sidewall 11, the eight partition plates 12 extend from the inner side surface of the inner sidewall 11 to the central axis of the columnar space 100 and converge at the central axis, the eight partition plates 12 divide the columnar space 100 evenly into eight storage spaces 110. The inner sidewall 11 and the partition plates 12 on its inner side provide a space for the expansion of the brewing material, the brewing effect will not be affected due to incapability of expansion of the brewing material. In addition, the partition plates 12 can form a plurality of flow channels at the inner side of the inner sidewall 11, so that the liquid can flow evenly in the space at the inner side of the inner sidewall 11 during brewing, and the brewing material is sufficiently soaked. A second end of the inner sidewall 11 of the bracket 10 is further provided with a plurality of end plates 14, the number of the end plates 14 is the same as the number of the partition plates 12, the end plates 14 and the partition plates 12 are correspondingly disposed, and the end plates 14 are perpendicular to the partition plates 12, the main function of the end plates 14 is to provide better support to the second end of the bracket 10. The end plates 14 are the base and reinforcing face of the partition plates 12, so that the partition plates 12 in the interior will not easy shrink and bend, and provide a fine working plane for heat sealing, so that the pressure is more uniform during packaging.

Referring to FIG. 1 and FIG. 2, a first end of the bracket 10 of the beverage capsule of the present embodiment, i.e., the first end of the inner sidewall 11, is provided with a first filter paper 21 and a first sealing film 31 for covering the first opening 101, the first sealing film 31 is located on an outer side of the first filter paper 21, and a second end of the bracket 10, i.e., the second end of the inner sidewall 11, is provided with a second filter paper 22 and a second sealing film 32 for covering the second opening 102, the second sealing film 32 is located on an outer side the second filter paper 22. The first sealing film 31 and the first filter paper 21 are provided at the first end of the inner sidewall 11 by heat sealing, and the second sealing film 32 and the second filter paper 22 are provided at the second end of the inner sidewall 11 by heat sealing, the material and shape of the inner sidewall 11 facilitate the heat sealing. The first sealing film 31 and the second sealing film 32 can be fiber films or other conventional and applicable film materials, which can be easily punctured. The first filter paper 21 and the second filter paper 22 may be any conventional and applicable filter papers, which can satisfy the filtration of the brewing material. The first sealing film 31 and the second sealing film 32 can also be translucent films, so that an inspector can quickly and sufficiently inspect the yield of heat sealing by observing the degree of adhesion between the end plates 14 and the second sealing film 32, and improve the efficiency of inspection after heat sealing.

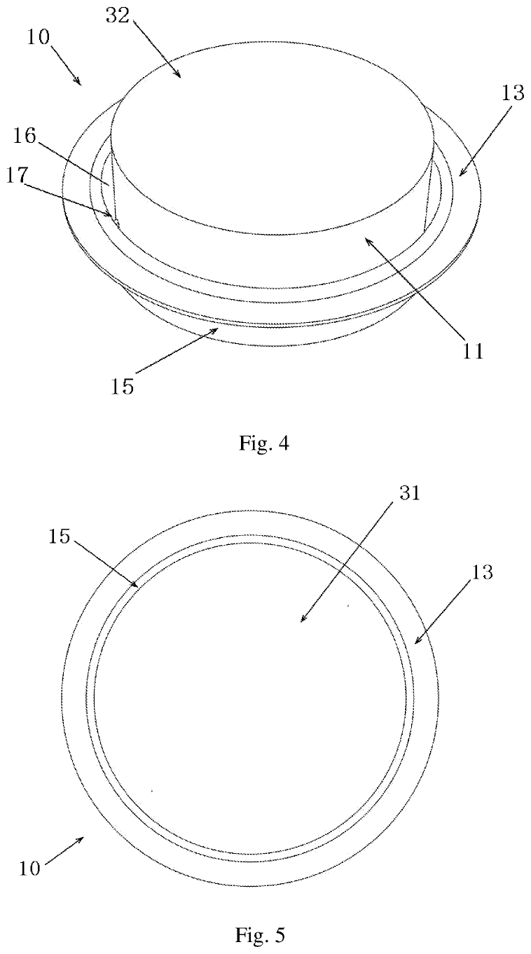

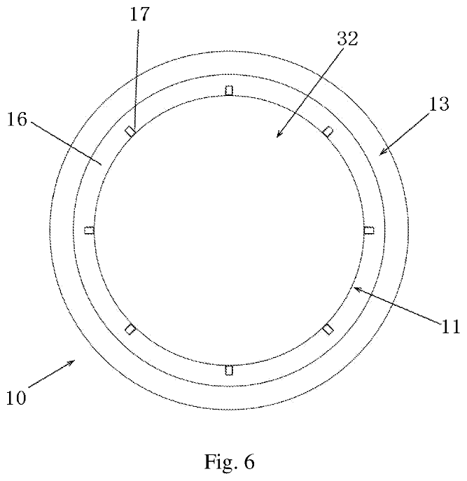

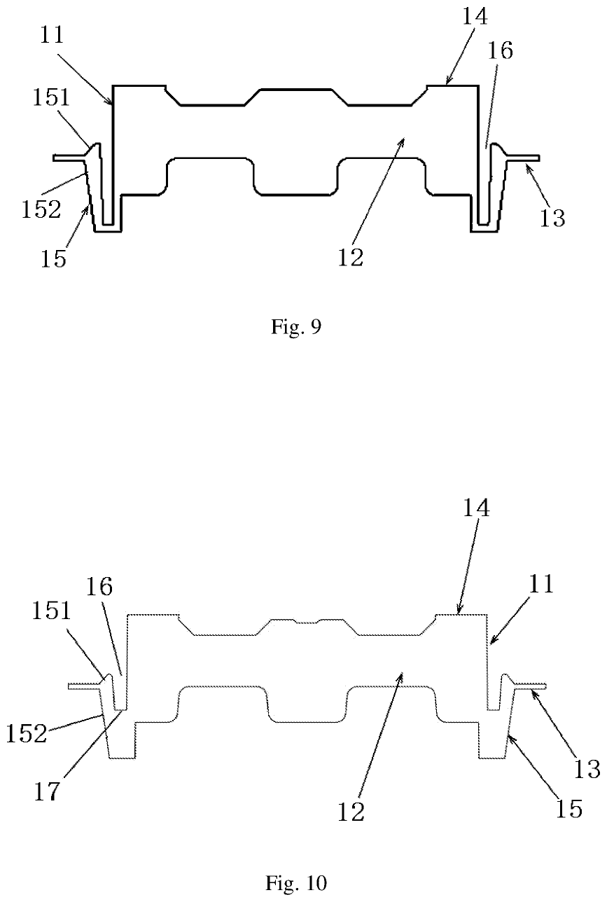

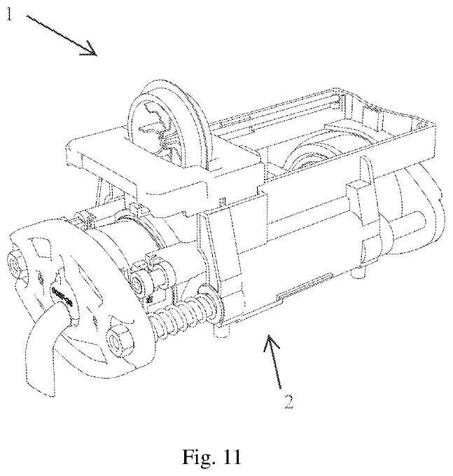

Referring to FIG. 2, FIG. 4, FIG. 6, and FIG. 8 to FIG. 10, in the present embodiment, the bracket 10 further includes an outer sidewall 15, the outer sidewall 15 first extends outward from one end of the inner sidewall 11 and then extends toward another end of the inner sidewall 11. An annular groove 16 is formed between the inner sidewall 11 and the outer sidewall 15, an annular block piece 13 is further formed on an outer side of the outer sidewall 15. The bracket 10 further includes a plurality of rib plates 17 formed by extending outward from an outer surface of the inner sidewall 11, each rib plate 17 corresponds to a partition plate 12, i.e., the rib plates 17 are one-to-one corresponding to the partition plates 12, the rib plates 17 are located in an extending direction of the partition plates 12, the plurality of rib plates 17 is located in the annular groove 16. This structural design enables the beverage capsule to be processed by molds with upper and lower structures without increasing the material, wall thickness and production costs, which not only improves the production quality, but also facilitates removal of the bracket from the mold for better and more efficient injection molding. The presence of the rib plates 17 can enable the annular block piece 13 to withstand higher pressure, e.g., brewing pressure of 19 bar, without being easily deformed during brewing. In addition, since the top and bottom have sufficient planes for heat sealing, the requirement of contact areas for heat sealing of the sealing films can be met at the same time. Adequate heat sealing enables the capsule to withstand higher pressure during brewing, so as to achieve a better brewing effect. The design of the annular groove 16 between the inner sidewall 11 and the outer sidewall 15 increases the toughness of the bracket 10 and the pressure-bearing capacity along the edges, making it easier for a positioning tool to clamp the capsule when the capsule is sealed in the brewing chamber without causing damage to the capsule casing under pressure. In addition, referring to FIG. 9 and FIG. 10, a portion of the outer sidewall 15 located above the annular block piece 13 and a portion of the outer sidewall 15 located below the annular block piece 13 have matching tapered surfaces 151, 152. This structural design makes the beverage capsule having a same size as the contacting front and rear rings of the sealing chamber during brewing, the sealing chamber will not form a gap due to different front and rear shapes of the capsule.

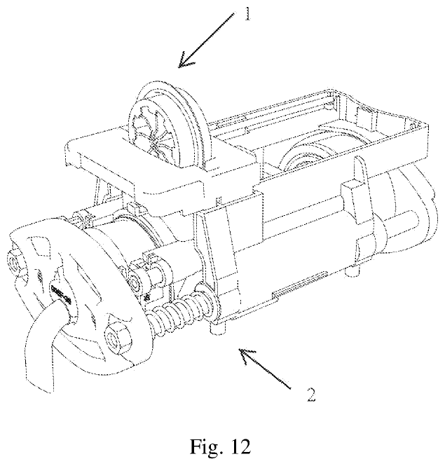

FIG. 11 and FIG. 12 show a schematic diagram of the beverage capsule 1 of the present application in use with a brewing system 2. As shown in the figures, the beverage capsule 1 can be loaded into the brewing system 2 in a forward or reverse direction, when placing the beverage capsule in the chamber, a user does not need to distinguish the front and the back, it can be placed in any direction, since the back pressure exerted on the front and back of the beverage capsule is the same, the brewing effect will not be affected due to uneven pressure, the brewing system 2 will use a needled disc to pierce through the first sealing film 31 and the second sealing film 32, and then hot water is injected for brewing, the first filter paper 21 and the second filter paper 22 play the role of filtering. The brewing liquid enters uniformly from the punctured holes of the second sealing film 32 at the second end of the beverage capsule, and flows out from the middle of the first sealing film 31 at the first end, the first filter paper 21 and the second filter paper 22 can sufficiently filter the brewing material and effectively filter impurities in the water, making the brewed liquid smooth and fine.

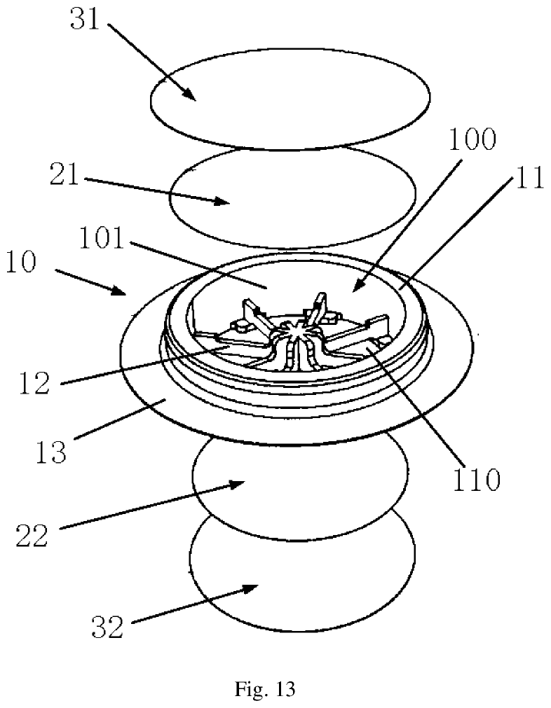

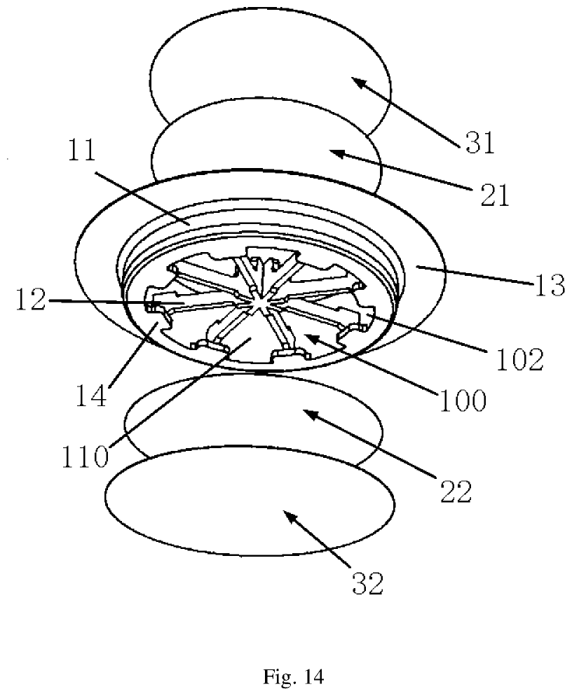

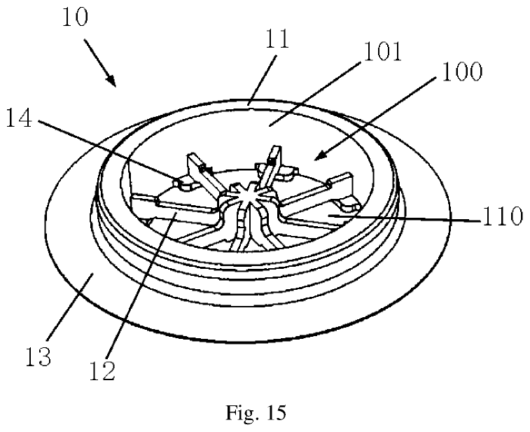

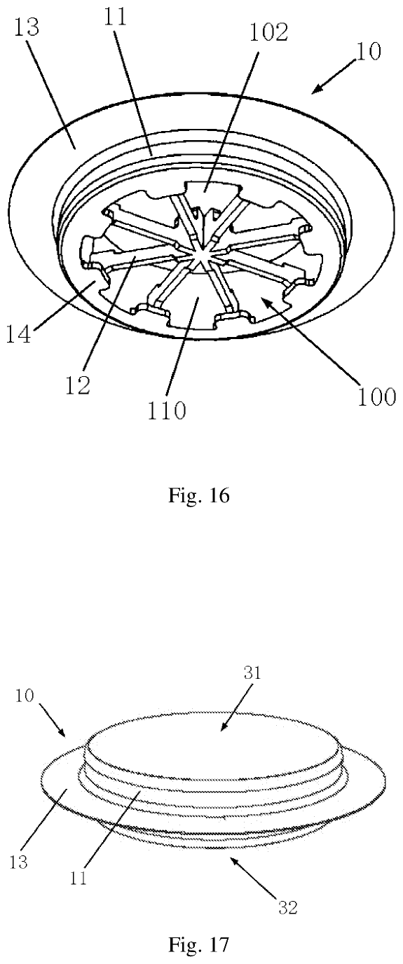

Referring to FIG. 12 to FIG. 17, the figures are schematic diagrams of another embodiment of the beverage capsule of the present application. The structure of the beverage capsule of this embodiment is similar to the structure of the embodiment of the beverage capsule shown in FIG. 1 to FIG. 12, except that in the embodiment shown in FIG. 12 and FIG. 17, the annular block piece 13 of the bracket 10 is formed by directly extending outward from a surface of the outer side of the inner sidewall 11, the annular block piece 13 may be formed in the middle of the inner sidewall 11, the inner sidewall 11 and the annular block piece 13 may be integrally formed. The annular block piece 13 provides a position for fixing the beverage capsule, which facilitates the positioning of the beverage capsule in the brewing system. The annular block piece 13 also provides a sealing end face, when the beverage capsule is placed in the brewing system, the sealing ring of the brewing system is pressed against the annular block piece 13, a sealed space can be formed on one side of the annular block piece 13 to facilitate soaking of the brewing material in the interior of the inner sidewall 11. The design of the annular block piece 13 and the inner sidewall 11 can also ensure that when the brewing chamber is opened, the used beverage capsule can fall downward and fall down accurately.

The beverage capsule of the present application is made of a degradable material. The bracket 10, the first filter paper 21, the second filter paper 22, the first sealing film 31, and the second sealing film 32 can all be degraded after the beverage capsule is brewed, so that no follow-up process is required as it can be degraded naturally.

In the beverage capsule of the present application, since the inner sidewall 11 of the bracket 10 and the partition plates 12 in the interior can play a supporting role, an inner space is sufficiently reserved, the brewing material in the interior is prevented from being squeezed during packaging and transportation, which affects the brewing effect. The inner sidewall 11 of the bracket 10 and the partition plates 12 in the interior make the brewing material evenly distributed in the columnar space 100, there will be no accumulation and squeezing after the brewing material is expanded, liquid can fully flow in and soak the material. It can also make the surface of the brewing material flat when it is expanded, so that the sealing films can smoothly touch the surface of the needled disc, achieving an ideal piercing effect, the expanded brewing material will not block the punctured holes, and the bracket itself does not touch the needled disc, and will not affect the puncture. The partition plates 12 in the inner sidewall 11 plays a role of draining the liquid during the brewing process, so that the liquid can circulate in the spaces separated by the partition plates 12, sufficiently soak the material in the spaces on the inner side of the inner sidewall 11, it will not lead to an unstable liquid flowing path due to uneven shape of the brewing material, and can achieve the best extraction effect. The inner sidewall 11 and the partition plates 12 provide support for the first filter paper 21 and the second filter paper 22, and underlay the filter papers so that the brewing material will not affect the filtering effect due to squeezing of the filter papers.

The embodiments of the present patent application are described above with reference to the accompanying drawings, but the present patent application is not limited to the above-mentioned specific embodiments. The above-mentioned specific embodiments are only illustrative, and not restrictive. Under the enlightenment of the present patent application, a person having ordinary skill in the art can come up with many forms without departing from the purposes of the present patent application and the scope of protection of the claims, which are all protected by the present patent application.

What is claimed is:

1. A beverage capsule, characterized in that it comprises a bracket, the bracket has a first opening and a second opening, the bracket includes an inner sidewall and a plurality of partition plates located on an inner side of the inner sidewall, the inner side of the inner sidewall forms a columnar space, the first opening is formed at one end of the columnar space, the second opening is formed at another end of the columnar space, the partition plates divide the columnar space evenly into a plurality of storage spaces, the storage spaces are filled with a brewing material, a first end of the bracket is provided with a first filter paper and a first sealing film for covering the first opening, the first sealing film is located on an outer side of the first filter paper, a second end of the bracket is provided with a second filter paper and a second sealing film for covering the second opening, the second sealing film is located on an outer side of the second filter paper;
the partition plates extend from a surface of the inner side of the inner sidewall to a central axis of the columnar space, or the partition plates extend from the surface of the inner side of the inner sidewall to the central axis of the columnar space and converge at the central axis;
the second end of the bracket has a plurality of end plates being formed by extending inward from an end face of the inner sidewall, each of the end plates corresponds to one of the partition plates and is connected to the partition plate in one-piece, the end plate is perpendicular to the partition plate;
the bracket further comprises an outer sidewall, the outer sidewall first extends outward from one end of the inner sidewall and then extends to another end of the inner sidewall, an annular groove is formed between the inner sidewall and the outer sidewall, an annular block piece is further formed on an outer side of the outer sidewall;
the bracket further comprises a plurality of rib plates formed by extending outward from an outer surface of the inner sidewall, each of the rib plates corresponds to one of the partition plates, the plurality of rib plates is located in the annular groove.

2. The beverage capsule according to claim 1, characterized in that the brewing material is a Chinese medicine in pieces, an herbal material or tea, and the herbal material includes one or more of a root, a stem, a leaf, a flower and a fruit of a plant.

3. The beverage capsule according to claim 1, characterized in that the first sealing film and the second sealing film are fiber films.

4. The beverage capsule according to claim 1, characterized in that the first sealing film and the second sealing film are translucent films.

5. The beverage capsule according to claim 1, characterized in that a portion of the outer sidewall located above the annular block piece and a portion of the outer sidewall located below the annular block piece have matching tapered surfaces.

6. A beverage capsule, characterized in that it comprises a bracket, the bracket has a first opening and a second opening, the bracket comprises an inner sidewall and a plurality of partition plates located on an inner side of the inner sidewall, the inner side of the inner sidewall forms a columnar space, the first opening is formed at one end of the columnar space, the second opening is formed at another end of the columnar space, the partition plates divide the columnar space evenly into a plurality of storage spaces, the storage spaces are filled with brewing material, a first end of the bracket is provided with a first filter paper and a first sealing film for covering the first opening, the first sealing film is located on an outer side of the first filter paper, a second end of the bracket is provided with a second filter paper and a second sealing film for covering the second opening, the second sealing film is located on an outer side the second filter paper;
the partition plates extend from a surface of the inner side of the inner sidewall to a central axis of the columnar space, or the partition plates extend from the surface of the inner side of the inner sidewall to the central axis of the columnar space and converge at the central axis;
the second end of the bracket has a plurality of end plates formed by extending inward from an end face of the inner sidewall, each of the end plates corresponds to one of the partition plates and is connected to the partition plate in one-piece, the end plate is perpendicular to the partition plate.

7. The beverage capsule according to claim 6, characterized in that the bracket further comprises an outer sidewall, the outer sidewall first extends outward from one end of the inner sidewall and then extends to another end of the inner sidewall, an annular groove is formed between the inner sidewall and the outer sidewall, an annular block piece is further formed on an outer side of the outer sidewall.

8. The beverage capsule according to claim 7, characterized in that the bracket further comprises a plurality of rib plates being formed by extending outward from an outer surface of the inner sidewall, each of the rib plates corresponds to one of the partition plates, the plurality of rib plates is located in the annular groove.

9. The beverage capsule according to claim 6, characterized in that the first sealing film and the second sealing film are translucent films.

10. The beverage capsule according to claim 7, characterized in that a portion of the outer sidewall located above the annular block piece and a portion of the outer sidewall located below the annular block piece have matching tapered surfaces.
